(19) United States
(12) Patent Application Publication
(10) Pub. No.: US 2019/0142712 A1
(43) Pub. Date: May 16, 2019

(54) Dentifrice To Promote Proper Tooth Brushing Techniques

(71) Applicant: Cody Neill, Lakewood Ranch, FL (US)

(72) Inventor: Cody Neill, Lakewood Ranch, FL (US)

(21) Appl. No.: 15/810,278

(22) Filed: Nov. 13, 2017

(51) Int. Cl. A61K 8/11; A61Q 11/00

(52) U.S. Cl. CPC A61K 8/11; A61K 6/0002; A61Q 11/00

(57) ABSTRACT

A dentifrice is provided for promoting proper tooth brushing and rinsing techniques. The dentifrice includes a carrier material, such as a toothpaste or gel, having persistent particles mixed therein. The persistent particles are resilient to friction during tooth brushing such that the particles are visually distinguishable from the carrier material when one expels the dentifrice from their mouth. The particles act as an indication to continue brushing and signal when rinsing is complete. The persistent particles may further resemble the shape of bacteria to act as a teaching tool to educate parents and children about the caries process, tooth decay, and cavity formation.

### BACKGROUND OF THE INVENTION

Dentifrice is generally known as a paste, gel, liquid, or powder for cleaning teeth. Dentifrice is currently available in many different types and brands with various compositions to address different teeth cleaning needs, including food debris removal, plaque removal, teeth whitening, preventing tooth decay, and decreasing tooth sensitivity. While the composition of dentifrice plays an important role in teeth cleaning, proper brushing and rinsing techniques are paramount to preventing tooth decay and cavity formation. Proper brushing techniques should be taught in the early stages of life (i.e. childhood) to instill good brushing habits to maintain oral hygiene. Likewise, proper rinsing techniques should also be taught as rinsing exposes the teeth to fluoride and removes toxins from the mouth that may otherwise be harmful if ingested. However, it is estimated that 60% of the United States population does not know how to teach their children proper brushing and rinsing techniques nor do the parents know the causes of cavities or the caries process. In addition, the parents may have a difficult time educating their children about the caries process, bacteria accumulation, and cavity formation associated with poor brushing and rinsing techniques.

One primary teaching point to a proper tooth brushing technique is the recommended brushing time. The American Dental Association recommends a minimum two-minute brushing time twice per day. Unfortunately, children often get bored during the two-minute time period and finish brushing prematurely. Toothbrushes having cartoon characters, lights, or other attention grabbing features have been developed to keep a child engaged throughout the brushing process. However, the child may lose interest and rush through brushing. Although child dentifrices exist to encourage brushing (e.g., dentifrices with a pleasing taste), these dentifrices do not encourage or teach proper brushing techniques nor educate the parent or child about the importance of proper brushing techniques.

Thus, there exists a need for a dentifrice and method to promote proper brushing and rinsing techniques. There further exists a need for a dentifrice to help educate a parent and child about the caries process, tooth decay, and cavity formation associated with poor brushing and rinsing techniques.

### FIELD OF THE INVENTION

The present invention generally relates to the field of dentifrices for teeth cleaning, and more particularly to a new and useful dentifrice to promote proper tooth brushing techniques.

### SUMMARY OF THE INVENTION

The general purpose of the dentifrice, described subsequently in greater detail, is to provide a dentifrice which has many novel features that results in the teaching and adoption of proper tooth brushing techniques, which is not anticipated, rendered obvious, suggested, or even implied by prior art, either alone or in combination thereof.

The dentifrice generally includes a carrier material and a plurality of persistent particles in the carrier material. The persistent particles are resilient to friction when subjected to tooth brushing and visually distinguishable from the carrier material when the dentifrice is expelled from one's mouth to act as an indication to continue tooth brushing and signal when rinsing is complete. The persistent particles may be made of a resilient insoluble, non-toxic, and non-abrasive material such as polymeric tinsel or glitter to maintain the general shape of the particles throughout the brushing process. The particles may be visually non-distinguishable from the carrier material prior to brushing to not stand out when the dentifrice is dispensed on a toothbrush. The concentration of the particles in the carrier material may further require the tooth brusher to properly brush for two to three minutes before all of the particles are expelled from the tooth brusher's mouth to ensure the tooth brusher brushes for the recommended brushing time.

A method is also provided for using the dentifrice to promote proper tooth brushing techniques. The method includes brushing one's teeth with the dentifrice described herein including: 1. expelling the dentifrice from one's mouth; 2. identifying if any persistent particles are in the expelled dentifrice; 3. continuing to brush and expel the dentifrice if any persistent particles are identified in the expelled dentifrice; and 4. rinsing the dentifrice from one's mouth when no persistent particles are identified in the expelled dentifrice. The method may further include educating the one about bacteria associated with tooth decay using the persistent particles as a teaching tool.

Thus has been broadly outlined the more important embodiments and features of the dentifrice so that the detailed description thereof that follows may be better understood and in order that the present contribution to the art may be better appreciated.

Advantages of embodiments of the present dentifrice, along with various novel features that characterize the invention are particularly pointed out in the claims forming a part of this disclosure. For better understanding of the various embodiments of the dentifrice, and its operating advantages attained by its uses, refer to the accompanying drawings and description.

### BRIEF DESCRIPTION OF THE DRAWINGS

### Figures

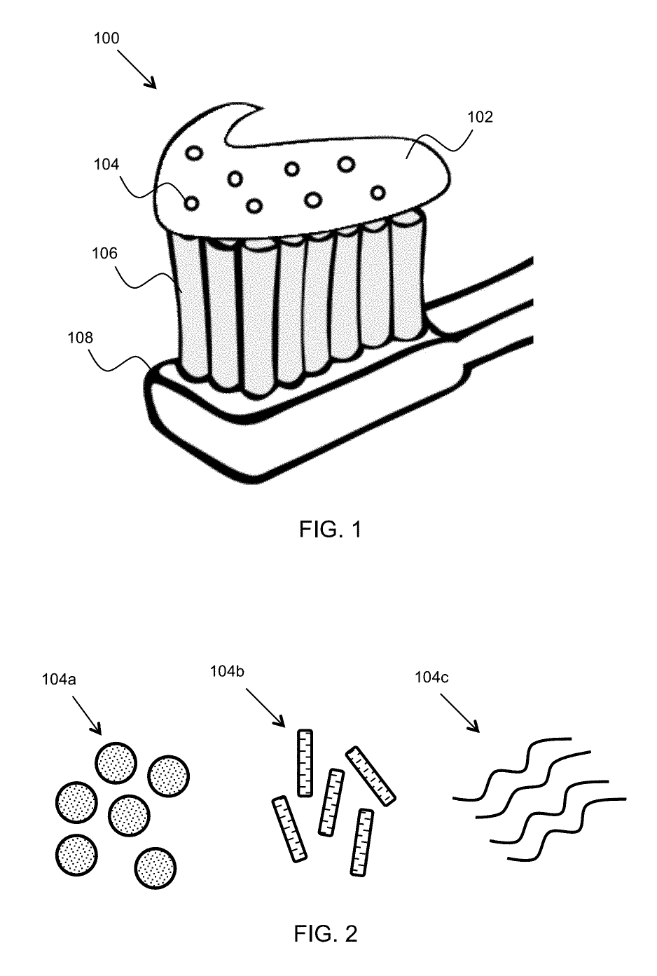

FIG. 1 is a perspective view of a dentifrice shown dispensed on bristles of a toothbrush in accordance with embodiments of the invention.

FIG. 2 depicts several bacteria shaped persistent particles in accordance with embodiments of the invention.

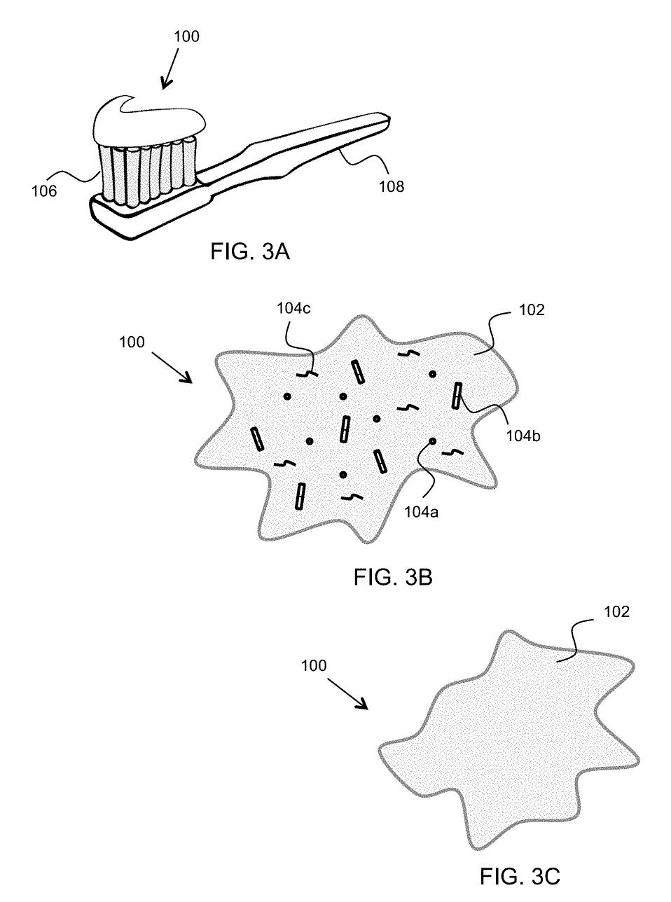

FIGS. 3A-3C visually depicts a method of using the dentifrice in accordance with embodiments of the invention, where FIG. 3A depicts the dentifrice prior to brushing, FIG. 3B depicts expelled dentifrice having persistent particles, and FIG. 3C depicts expelled dentifrice having no persistent particles.

### DETAILED DESCRIPTION OF THE INVENTION

The present invention has utility as a dentifrice and method of use to promote proper tooth brushing and rinsing techniques, especially for children. The dentifrice is additionally useful as a teaching tool to educate parents and children about cavity formation and oral hygiene in general. The following description of various embodiments of the invention is not intended to limit the invention to these specific embodiments, but rather to enable any person skilled in the art to make and use this invention through exemplary aspects thereof.

It is to be understood that in instances where a range of values are provided that the range is intended to encompass not only the end point values of the range but also intermediate values of the range as explicitly being included within the range and varying by the last significant figure of the range. By way of example, a recited range from 1 to 4 is intended to include 1-2, 1-3, 2-3, 2-4, and 1-4.

With reference now to the drawings, FIG. 1 illustrates an inventive embodiment of a dentifrice 100 shown dispensed on bristles 106 of a toothbrush 108. The dentifrice 100 is generally composed of materials that clean one's teeth and promote proper tooth brushing techniques. In particular, the dentifrice 100 includes a carrier material 102 and a plurality of persistent particles 104 in the carrier material 102 that are resilient to friction when subjected to tooth brushing and visually distinguishable from the carrier material 102 when the dentifrice is expelled from one's mouth to act as an indication to continue brushing and signal when rinsing is complete.

The carrier material 102 is generally composed of one or more materials configured to clean one's teeth and provide the vehicle for the persistent particles 104. The carrier material 102 may be a paste, liquid, gel, or powder for cleaning teeth known in the art. In a specific embodiment, the carrier material 102 is a children's toothpaste.

The plurality of persistent particles 104 may be made of one or more materials that are resilient to friction when subjected to tooth brushing and visually distinguishable from the carrier material when expelled from one's mouth. In other words, the persistent particles 104 are made of one or more materials capable of withstanding the friction generated during tooth brushing such that the particles generally maintain their original shape throughout brushing. The resilient material(s) of the persistent particles 104 may further be insoluble in the carrier material 102, or at least insoluble in the majority ingredient of the carrier material 102, so the particles 104 do not dissolve during brushing or while the dentifrice 100 is stored. The material(s) of the particles 104 may further be non-toxic so as not to harm to the tooth brushers general health if the particles 104 are accidentally ingested. In addition, the material(s) of the particles 104 may be non-abrasive to avoid scratching, cutting, or irritating the tooth brushers gums, teeth, lips, or cheeks. Conversely, the material(s) of the particles 104 may be slightly abrasive, or have an abrasive surface, to act as a polishing agent. In a specific inventive embodiment, the particles 104 are made of insoluble, non-toxic, and non-abrasive polymeric tinsel or glitter. In other embodiments, the particles 104 are made of a natural material or glitter that is minimally soluble in the carrier material 102. In some embodiments, the minimum solubility is characterized by the persistent particles 104 completely dissolving in the carrier material 102 within 1 month to 1 year. In other embodiments, the minimum solubility is characterized by the persistent particles 104 completely dissolving in the carrier material 102 in 1 to 3 years. In a further embodiment, the minimum solubility is characterized by the persistent particles 104 completely dissolving in the carrier material 102 in greater than 3 years.

In a specific inventive embodiment, with reference to FIG. 2, the persistent particles 104 are designed, manufactured, or otherwise formed to resemble the shapes of bacteria, illustratively including bacilli 104a (ball-shaped), cocci 104b (rod shaped), and spirochete 104c (spiral or half-moon shaped). The bacteria shaped persistent particles (104a, 104b, 104c) provides a pathway for parents to open a dialogue and educate their children about bacteria's role in the caries process, tooth decay, cavity formation, types of bacteria found in the mouth, and the types of matter bacteria consume and proliferate amongst (e.g., sugar), all while their child brushes their teeth. The parents likewise become educated on these topics to become a better mentor to their children's oral hygiene. In addition, the bacteria shaped particles (104a, 104b, 104c) keep the children engaged and entertained during brushing, with the mentality of wanting to get “all of the bugs out” of their mouths. The education, engagement, and increased brushing time to “get all the bugs out” promotes good oral hygiene and instills proper brushing techniques at a young age. Furthermore, the child becomes knowledgeable about the types of food that are more likely to cause cavities, which may promote a healthy diet, or at least increase the frequency of brushing after eating such foods. It should be appreciated that although only three bacteria shaped particles (104a, 104b, 104c) are shown in FIG. 2, the persistent particles 104 may resemble several other bacteria shapes (e.g., Streptococcus mutans, Treponema denticola, Porphyromonas gigivalis), or other shapes related to other educational subjects. Further, the variety of particle shapes in the dentifrice 100 may vary widely, where one product of dentifrice 100 may include persistent particles 104 all having one single shape, while another product of dentifrice 100 may include persistent particles 104 having two or more different shapes.

In addition to the shape of the persistent particles 104, the persistent particles 104 have dimensions and or properties that permit the particles 104 to be visually distinguishable from the carrier material 102 when the dentifrice 100 is expelled from one's mouth. Preferably, the dimensions of the persistent particles 104 are small enough so as to not irritate the gums, teeth, or cheeks during brushing, but large enough to be seen by the naked eye in the dentifrice 100 when expelled. In a particular embodiment, the persistent particles 104 have a minimum size in the range of 0.1 mm to 2 mm, where 0.1 mm is generally regarded as the smallest visual resolution of a human naked eye. In another embodiment, the persistent particles 104 have dimensions ranging from 0.1 mm-2 mm (length)×0.1 mm-2 mm (width). In a further embodiment, the persistent particles have a minimum diameter ranging from 0.1 mm-2 mm.

The persistent particles 104 may include other properties to be visually distinguishable from the carrier material 102 when the dentifrice 100 is expelled from one's mouth. For example, the persistent particles 104 may have a color different from a color of the carrier material 102 when expelled. The persistent particles 104 may have an albedo or reflectance different from an albedo or reflectance of the carrier material 102 when expelled. The persistent particles 104 may have both a color and albedo different from the carrier material 102 when expelled.

In a specific inventive embodiment, the persistent particles 104 are visually non-distinguishable from the carrier material 102 prior to brushing one's teeth such that the particles 104 do no stand out when the dentifrice 100 is dispensed onto a toothbrush. The carrier material 102, prior to brushing, may have a color, gradient, or visual pattern to camouflage the persistent particles 104 therein. The carrier material 102 may change color throughout the brushing process (common of many dentifrices) such that the persistent particles 104 are visually distinguishable from the carrier material 102 when the dentifrice 100 is expelled. For example, prior to brushing, the carrier material 102 and the persistent particles 104 may be green in color. When the dentifrice 100 is expelled, the carrier material 102 may be white, where the persistent particles 104 maintain their green color and are therefore visible in the expelled dentifrice 100.

In a specific inventive embodiment, the persistent particles 104 have glow in the dark properties. The persistent particles 104 may be coated, painted, or at least partially made with phosphorescent materials known in the art. The particles 104 are charged by exposure to light and then illuminate in the dark. The particles 104 with phosphorescence may be camouflaged in the carrier material 102 while the user brushes their teeth in the light, and upon turning off the lights the particles 104 illuminate in the dark. The use of particles 104 having phosphorescence improves the brushing experience and further encourages a user to brush their teeth.

In a particular inventive embodiment, the plurality of persistent particles 104 in a single serving size of dentifrice 100 exists in a quantity, mass percentage, or volume percentage that requires 2 to 3 minutes of proper brushing technique to remove all of the particles 104 from one's mouth. Therefore, with a proper brushing technique the tooth brusher brushes for at least the recommended 2-minute minimum brushing time. Longer brushing times, to remove all of the persistent particles 104, may occur if improper brushing techniques are used, which incentivizes the tooth brusher to learn and adopt a proper brushing technique. The single serving size of dentifrice 100 may be 0.2 gram to 0.3 grams. In a specific embodiment, the persistent particles 104 constitute 0.1% to 5% by volume in the carrier material 102.

The dentifrice 100 may be manufactured using techniques know in the art. The persistent particles 104 may be mixed in the carrier material 102 during the dentifrice 100 manufacturing process using a standard industrial mixing technique.

With reference to FIGS. 3A-3C, an embodiment of a method of using the dentifrice 100 is visually depicted. FIG. 3A illustrates the dentifrice 100, prior to brushing, dispersed on bristles 106 of a toothbrush 108. The persistent particles 104 are not shown in the pre-brush dentifrice 100, as the particles 104 are visually non-distinguishable from the carrier material 102 in this embodiment. The method begins by one brushing their teeth with the dentifrice 100. Subsequently, the dentifrice 100 is expelled from one's mouth. FIG. 3B illustrates how the dentifrice 100 may appear upon first expelling the dentifrice 100 from one's mouth. The carrier material 102 of the expellant is shown as a general blob of material, similar to what might appear in one's sink. Bacteria shaped persistent particles (104a, 104b, 104c) are shown scattered about the expelled carrier material 102. The presence of the particles (104a, 104b, 104c) indicates the recommended time for brushing has not been achieved and/or the tooth brusher is using an improper brushing technique. At this time, a parent, or other bystander, may optionally explain or educate the tooth brusher about the caries process, bacteria associated with tooth decay, or cavity formation using the persistent particles (104a, 104b, 104c) as a teaching tool. Finally, the dentifrice is rinsed from one's mouth when no persistent particles 104 appear in the expelled dentifrice 100 as shown in FIG. 3C. The tooth brusher may need to rinse several times until all of the particles 104 are removed from their mouth to further promote proper rinsing techniques.

### OTHER EMBODIMENTS

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A dentifrice to promote proper tooth brushing and expelling, comprising:
a carrier material;
a plurality of persistent particles in the carrier material that are resilient to friction when subjected to tooth brushing and visually distinguishable from the carrier material when the dentifrice is expelled from one's mouth to act as an indication to continue tooth brushing and signal when rinsing is complete; and
wherein the plurality of persistent particles are in the shape of bacterium including bacilli, cocci, and spirochete to educate one about bacteria associated with tooth decay to further promote proper tooth brushing techniques.

2. The dentifrice of claim 1 wherein the plurality of persistent particles are visually non-distinguishable from the carrier material prior to being subjected to tooth brushing.

3. The dentifrice of claim 1 wherein one or more of the plurality of persistent particles are in the shape of a bacterium.

4. The dentifrice of claim 3 wherein the shape of bacterium is selected from the group of bacilli, cocci, or spirochete.

5. The dentifrice of claim 1 wherein the carrier material is a paste, gel, or liquid.

6. The dentifrice of claim 1 wherein the plurality of persistent particles are made of an insoluble and non-toxic material.

7. The dentifrice of claim 6 wherein the plurality of persistent particles are made of a non-abrasive material.

8. The dentifrice of claim 7 wherein the material is polymeric tinsel or glitter.

9. The dentifrice of claim 6 wherein the plurality of persistent particles comprise a phosphorescent material that illuminates in the dark.

10. The dentifrice of claim 3 wherein the plurality of persistent particles have a minimum size in the range of 0.1 mm to 2 mm.

11. The dentifrice of claim 1 wherein the plurality of persistent particles in 0.2 grams to 0.3 grams of carrier material exists in a quantity that requires 2 to 3 minutes of brushing and expelling to remove all of the particles from one's mouth.

12. A method to promote proper tooth brushing and expelling, comprising:
a. brushing one's teeth with the dentifrice of claim 1;
b. expelling the dentifrice from one's mouth;
c. identifying if any persistent particles are in the expelled dentifrice;
d. continuing to brush and expel if any persistent particles are identified; and
e. rinsing the dentifrice from one's mouth when no persistent particles are identified in the expelled dentifrice.

13. The method of claim 12 further comprising, educating the one about bacteria associated with tooth decay using the persistent particles as a teaching tool.
